(19) United States
(12) Patent Application Publication
(10) Pub. No.: US 2007/0166706 A1
(43) Pub. Date: Jul. 19, 2007

(54) Method for detecting biomolecules

(76) Inventors: Alfred Nordheim, Tubingen (DE); Winfried Kammer, Tubingen (DE); Stefanie Weiss, Tubingen (DE); Giang Vuong, Tubingen (DE)

Correspondence Address: NATH & ASSOCIATES, 112 South West Street, Alexandria, VA, 22314, US

(21) Appl. No.: 10/527,055

(22) PCT Filed: Sep. 8, 2003
(86) PCT No.: PCT/EP03/09923

§ 371 Date: May 18, 2005

(30) Foreign Application Priority Data
Sep. 13, 2002 (DE) 102 43 303.8

(51) Int. Cl. C12Q 1/68; C12Q 1/37

(52) U.S. Cl. 435/6; 435/23

(57) ABSTRACT

The invention relates to a method for detecting biomolecules, particularly peptides, proteins, carbohydrates, glycoproteins, proteoglycans and/or nucleic acids by using a metal compound in the presence of at least one bifunctional agent.

The invention relates to a method for detecting biomolecules by means of a metal compound in the presence of at least one at least bifunctional agent.

Detection and characterization of biomolecules are of fundamental importance for biological research and clinical medicine. Detection and characterization methods for different biomolecules are regularly employed in particular in the search for mutation events and in the diagnostics of genetically caused disorders.

Biomolecules here mean in particular the group consisting of peptides, proteins, glycoproteins, proteoglycans, carbohydrates and nucleic acids.

The first step of detection, the fractionation of said molecules, currently involves using mostly one- or two-dimensional gel electrophoresis systems. Electrophoresis means a fractionation of charged particles under the influence of an electric field. It is possible to use for electrophoresis various support materials, inter alia agarose gels, cellulose acetate gels or polyacrylamide gels. Owing to the superior separating action, compared to agarose gels, preference is given to using polyacrylamide gels for protein characterization. After completion of the gel-electrophoretic separation, the biomolecules must be visualized on the support material. To this end, various visualization techniques exist, such as, for example, Coomassie Blue staining, fluorescent labeling, radiolabeling, ethidium bromide staining and silver staining. In all methods listed, there exist large differences with respect to sensitivity, the amount of time and material needed, as well as environmental compatibility and harmfulness to health of the reagents used and the waste produced. Thus, for example, Coomassie Blue staining is very easy to carry out but, in return, has very low sensitivity. Undesired radioactive or carcinogenic waste is produced especially in the case of radiolabeling or ethidium bromide staining. Fluorescent labeling has the disadvantage of needing a relatively complex apparatus. Silver staining is approx. one hundred times more sensitive compared with Coomassie Blue staining, with no radioactive or carcinogenic waste being produced and instrumental complexity being relatively low. Therefore, silver staining is currently the most frequently used staining method for visualizing proteins. However, silver staining has the decisive disadvantage of being very time consuming, if an appropriate sensitivity is desired. This, however, is a decisive disadvantage, especially in modern medical diagnostics and life science research.

The various silver staining methods can be divided in principle into two groups, depending on the silver compound used. A distinction is made between silver nitrate staining and silver diammine staining. For more details on this, see Electrophoresis 13, 429-439 (1992) by T. Rabilloud.

Another difference between the different visualization techniques and also within the various variations of silver staining is the availability of the molecules for further characterization methods, in particular examination by mass spectrometry. In the case of many visualization methods and previous silver stainings, the molecule is in a chemically modified form after detection and is therefore no longer available, or available only in a form inadequate for characterization, to an examination by mass spectrometry.

Independently of the exact staining method and process control, most silver stainings share the sequence of the steps fixing, sensitizing, silvering step (incubation with a solution containing silver ions), developing and stopping of the reaction.

The molecules are fixed by incubating the gels first with an acidic alcoholic solution. Subsequently, a sensitizing step is carried out which involves incubating said gels with reducing agents such as glutaraldehyde, DTT, dithionite or thiosulfate. Said reagents are responsible for reducing silver ions on the surface of the biomolecules to very small amounts of metallic silver and serve, in the developing step, as nuclei for further precipitation on silver (for more details on this, see Electrophoresis 11, 785-794 (1990) by T. Rabilloud). After a washing step in which excess reducing agent is removed, silver staining/silver impregnation of the gel is carried out by means of silver nitrate or silver diammine solution. After this silvering step, the gel is again washed. and then developed with a developing solution containing either formaldehyde and sodium carbonate or formaldehyde and citric acid. After completion of the developing reaction, the gel is incubated in a stopping solution in order to stop the developing action. Stopping solutions usually contain Tris/acetic acid, citric acid or complexing agents such as EDTA or EGTA, for example.

All currently known methods either have the disadvantage of being very lengthy, with silver staining lasting from >5 to 24 h, in order to achieve a relatively high sensitivity in the process, or they are comparatively short, i.e. <5 h, but are less sensitive in the process. There exist moreover relatively sensitive silver staining methods which, however, do not permit any satisfactory examination of the biomolecule by mass spectrometry following detection.

It is a substantial object of the invention to develop a rapid and, at the same time, very sensitive silver staining method which permits characterization of the biomolecules by mass spectrometry, following detection.

This object is achieved by the detection method of the invention, having the features of claim 1, and the kit having the features of claim 22. Preferred embodiments are illustrated in the dependent claims 2 to 21 and 23 and 24. The wording of all the claims is hereby incorporated by reference in the contents of the present description.

According to the invention, a method for detecting biomolecules, in particular peptides, proteins, glycoproteins, proteoglycans, carbohydrates and/or nucleic acids, by means of a metal compound involves the use of a bifunctional agent having a hydrophobic and a reducing moiety. It is also possible for the agent to have more than one hydrophobic moiety and/or more than one reducing moiety. It is likewise conceivable to use more than one at least bifunctional agent for detection.

According to the invention, the bifunctional agent is a molecule of the general formula X—R, with the moiety X of said bifunctional agent preferably being the reducing moiety.

In a preferred embodiment, the moiety X is in particular a linear or homo- and/or heterocyclic hydrocarbon.

The moiety X has preferably at least one hydroxyl group, at least one sulfhydryl group, at least one carbonyl group, at least one thiosulfate group and/or at least one unsaturated carbon-carbon bond.

In a particular embodiment, X is a molecule having antioxidative properties, preferably a vitamin, in particular from the group consisting of vitamin A, vitamin C and/or vitamin E. In a particular embodiment, the moiety X of the biofunctional agent is ascorbic acid.

According to the invention, R is the hydrophobic moiety of the bifunctional agent.

In a particular embodiment, R is a saturated hydrocarbon. It is also conceivable that R is an at least monounsaturated hydrocarbon. According to a preferred embodiment, R is an acyloxy radical of the general formula —O—CO—CₙH₍₂ₙ₊₁₎, where n is preferably 8-21, in particular 11-17 and particularly preferably 15.

In a particular embodiment, the bifunctional agent is ascorbyl palmitate. However, it is also conceivable for said bifunctional agent to be ascorbyl stearate, ascorbyl myristate or ascorbyl laurate.

According to the invention, the bifunctional agent may be present at a final concentration of from 10⁻⁵ to 1%, preferably from 10⁻⁴ to 0.1%, in particular 5×10⁻⁴ to 5×10⁻³%, during detection. In a particularly preferred embodiment, the final concentration of the bifunctional agent is 10⁻³%.

According to the present invention, the metal compound is a silver compound, in particular silver nitrate. In a different embodiment, said silver compound may also be a silver diammine.

The nucleic acids to be detected are preferably DNA or RNA.

In an embodiment of the method of the invention, the molecules to be detected are applied onto or into a support for detection. In the case of proteins, said support is preferably a polyacrylamide gel. In the case of DNA, said support is preferably agarose gels. In the case of RNA, the use of agarose gels or of polyacrylamide gels is conceivable, depending on the size of the RNA fragments. In another embodiment, the support material is a membrane, in particular a PVDF or nitrocellulose membrane. It is also conceivable for the support to be a microarray support, in particular a biochip. The method of the invention may also be used for staining proteins from cells which have been removed from a tissue by means of LCM (laser capture microdissection).

According to the invention, the method for detecting biomolecules comprises at least the following steps: first, the molecules are fixed on or in the support by incubating with a fixing solution, followed by washing the support with the molecules in at least one washing step with a first washing solution and subsequently with a second washing solution. In the subsequent metal compound step, the support material with the molecules fixed thereon or therein is incubated with a solution of the metal compound and washed with water of highest purity in the subsequent washing step. This is followed by the developing step comprising a developing solution and the final stopping step.

According to the method of the invention, the bifunctional agent may be used in the fixing step, in particular as an additive to the fixing solution. Said fixing solution may contain, besides the bifunctional agent, 20-50%, in particular 40%, ethanol.

According to one embodiment, the bifunctional agent is used in an at least partially alcoholic solution. Said alcoholic solution is preferably an ethanolic solution, in particular one of absolute ethanol.

In the method of the invention, a complexing agent, in particular EDTA, may be used as a component of the developing solution in the developing step. In another embodiment, the complexing agent used may also be EGTA.

Further components of the developing solution, in addition to the complexing agent, may also be sodium carbonate, sodium thiosulfate and/or a reducing reagent, preferably from the group of aldehydes. In a particular embodiment of the invention, the reducing reagent is formaldehyde.

In the method of the invention, detection of the biomolecules may be followed by further characterization, preferably a study by mass spectrometry, in particular identification of said biomolecules by means of MALDI-MS or by ESI-MS.

The invention furthermore comprises a kit for detecting biomolecules, which comprises at least one at least bifunctional agent. The bifunctional agent present in the kit has at least one hydrophobic and at least one reducing moiety. In one embodiment of said kit, the bifunctional agent is present in the fixing solution. The kit of the invention furthermore comprises at least one feature of claims 2 to 11, which relate to the bifunctional agent and which have already been illustrated above.

The kit moreover comprises the feature of claim 19 which relates to the developing step. In connection with the kit, express reference is made to this illustration.

The method of the invention is illustrated below by the detailed description of particular embodiments and by figures. In said embodiments, individual features of the invention may be implemented alone or in combination with other features. The particular embodiments described serve merely the purpose of illustrating the invention and of better understanding thereof and are in no way to be construed as being limiting.

Otherwise, the invention is depicted on the basis of the figures in which:

FIG. 1 depicts the sensitivity of protein staining methods.

FIG. 2 depicts selected protein spots for identification by mass spectrometry by way of example of a 2D gel stained with colloidal Coomassie. In the case of the other staining reactions, the corresponding spots were selected for MS identification.

FIG. 3 depicts MALDI-MS sequence coverage in %

FIG. 4 (consisting of parts 4A, 4B and 4C) depicts protein identification by ESI-MS after tryptic digest, illustrated in a table consisting of parts A, B and C.

### DESCRIPTION OF THE FIGURES

FIG. 1 depicts the different sensitivities of protein staining of the three known protein staining methods according to Hochstrasser (see experimental section), Amersham Biosciences Plus One Silver Staining Kit (#17-1150-01) and the fluorescence staining method SYPRO Ruby from Bio-Rad (# 170-3125), as well as the novel staining method of the invention. The novel method for detecting biomolecules is more sensitive, by at least a factor of 30, than the previously known methods according to Hochstrasser and Amersham Biosciences, and in addition distinctly more sensitive than labeling by a fluorescent dye according to the method using SYPRO Ruby. The conventional silver staining methods, for example the method according to Hochstrasser and Amersham Biosciences, mostly have the disadvantage that they the biomolecules to be detected, after detection, being no longer or only inadequately accessible to a subsequent examination by mass spectrometry, owing to the use of glutaraldehyde as sensitizer in the sensitizing step. The inventive method for detecting biomolecules, however, uses, instead of glutaraldehyde, a bifunctional molecule which does not have the disadvantages of glutaraldehyde and enables the biomolecules to be identified by mass spectrometry after detection. In order to prove that proteins can be identified with the aid of the inventive method means of mass spectrometry methods after detection, a comparative experiment was carried out. The 15 protein spots labeled in FIG. 2 were selected from four 2D gels which were stained in parallel by four different staining methods. The four staining methods were firstly the classical colloidal Coomassie (G250) staining which is known to be compatible with mass spectrometry. The second method is the silver staining method of the invention, which is described in more detail in the experimental section. The third method is the staining according to Hochstrasser, using glutaraldehyde. The fourth method is the staining using the Plus One Silver Staining Kit from Amersham Biosciences, likewise using glutaraldehyde.

Following the different staining methods, the individual protein spots were extracted and then examined both by means of MALDI-MS and ESI-MS. The results of these protein identifications are depicted in table 1 and table 2.

FIG. 3 depicts the sequence coverage in percent for spots No. 1 to No. 15 after a MALDI-MS for the four different protein staining methods. The average sequence coverage for each method was determined. Values in parentheses were not included in forming the average, since the peptide masses found could originate from different isoforms of a protein and thus it is not possible to determine an unambiguous value for sequence coverage. In this determination by mass spectrometry, the silver staining method of the invention, with 19% sequence coverage, proved to be superior even to the classical staining with colloidal Coomassie (17.1% sequence coverage). This clearly indicates the suitability of the detection method of the invention for identification of the biomolecules by mass spectrometry, carried out following detection. The results of the two staining methods according to Hochstrasser (6.3% sequence coverage) and the staining by means of the Amersham Biosciences Kit (9% sequence coverage) clearly indicate an impairment of identifiability, which can be attributed to the use of glutaraldehyde. The overall very low sequence coverage values result from the low amounts of protein used for this comparative experiment.

FIG. 4 depicts a representation of the amino acids recovered of selected peptides from the individual protein spots and determination of sequence coverage. Detection by means of ESI-MS also indicates clearly the suitability of the detection method of the invention in comparison with the method according to Hochstrasser or Amersham Biosciences. The sequence coverage of the novel silver staining method was, like that of the colloidal Coomassie staining, 67%, in contrast to 35% in the case of the two other methods (Hochstrasser and Amersham Biosciences).

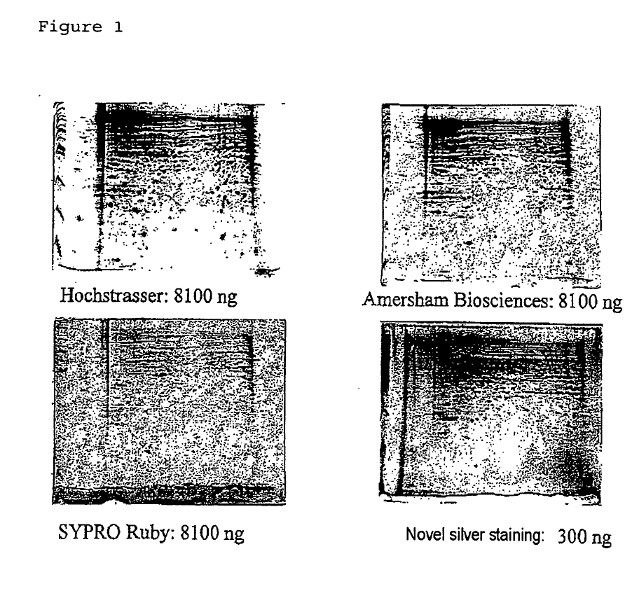

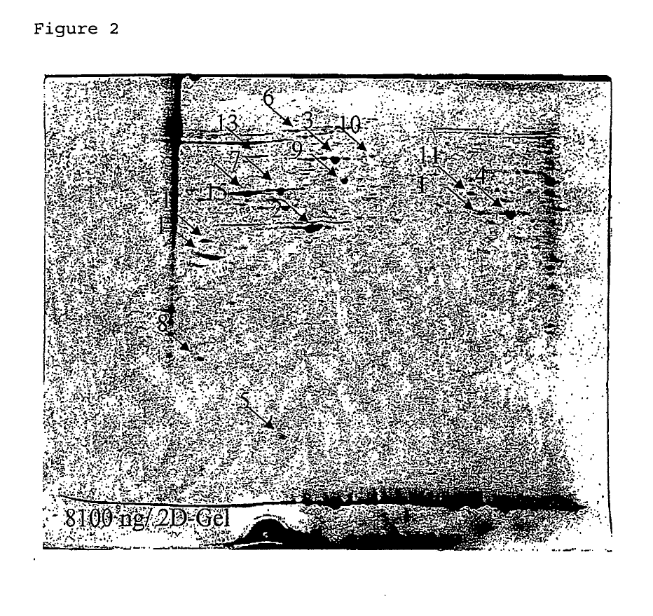

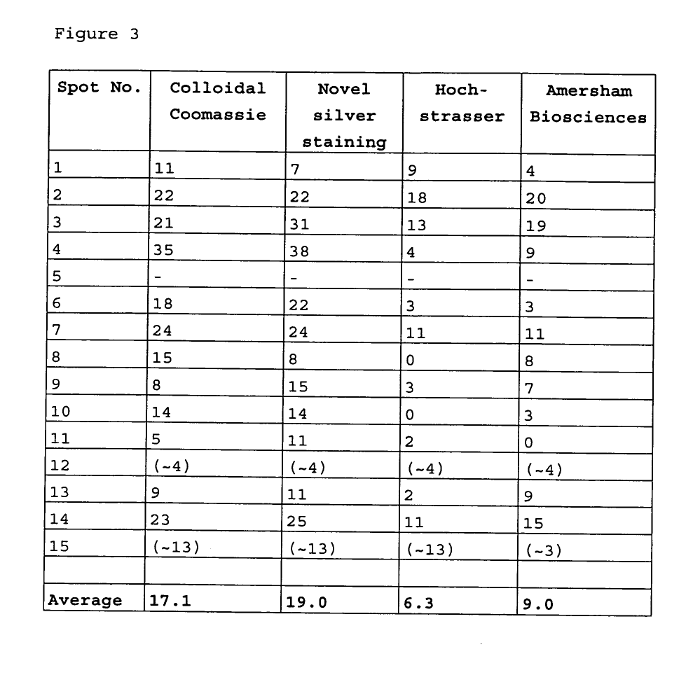

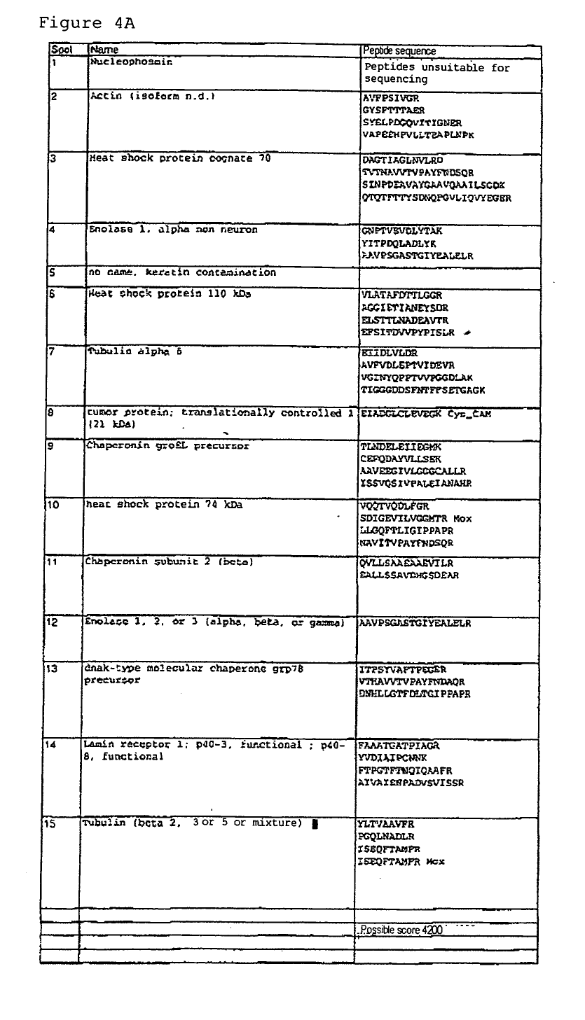

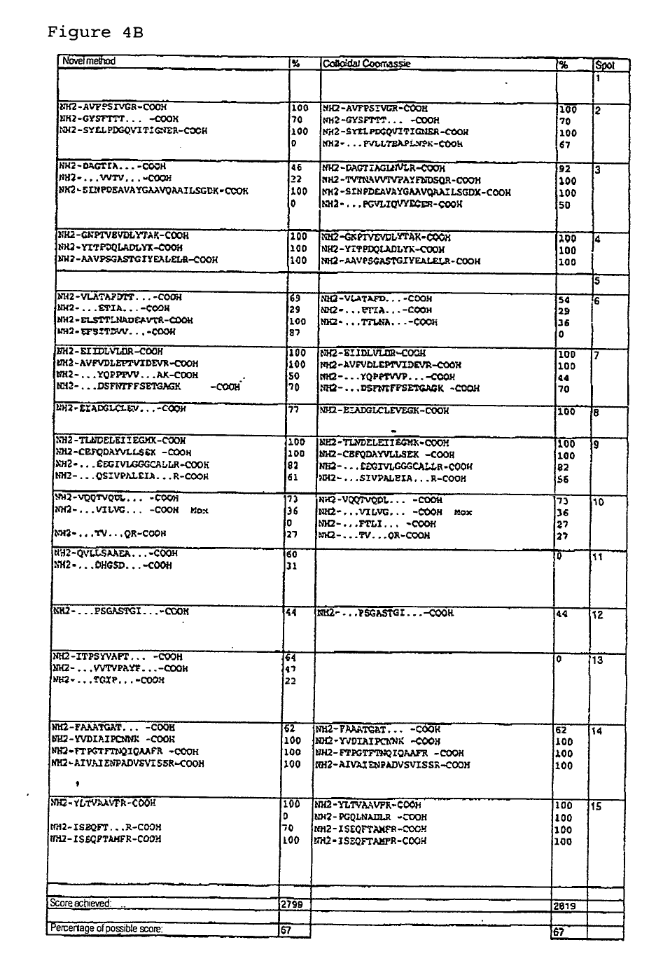

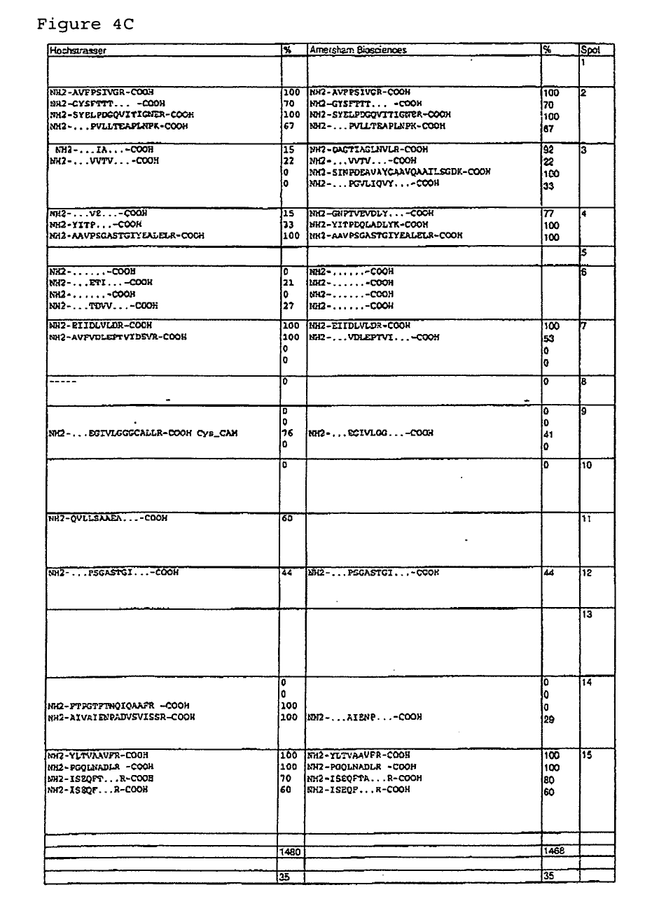

### Experimental Section

The protein mixture used for the different stainings was obtained from murine embryonic stem cells in the following manner: 10 million cells were centrifuged in an Eppendorf reaction vessel and the pellet was subsequently lysed with a lysis buffer consisting of 9 M urea, 4% CHAPS (cholamidopropyl-dimethylammononiopropanesulfonate), 1% DTT (dithiothreitol), 1% Pharmalyte (pH 3-10) and 0.001% Bromphenol Blue. The concentration of the protein solution was determined according to Bradford [Bradford, M. Analyt. Biochem. 72, 248-254, 1976).

Silver Staining of Biomolecules:

1. Method of the Invention

The biomolecules to be stained are in the polyacrylamide gels and are agitated on a horizontal shaker during the entire staining process. The solutions may be changed between the individual staining steps by removing by suction the no longer needed solutions and adding fresh solutions or by transferring the gels to new staining dishes.

1st Step: Fixing of the Biomolecules

The polyacrylamide gel containing the biomolecules is introduced into a fixing solution consisting of 40% strength ethanol and 10⁻³% ascorbyl palmitate. Ascorbyl palmitate was added in the form of a solution of ascorbyl palmitate in absolute ethanol. The fixing process takes 30 minutes.

2. Washing Steps:

The gels are washed first with a 20% strength and then with a 10% strength ethanol solution. The time in each case is 15 minutes.

3. Silvering Step:

After removing the washing solution, the gels are incubated in a 0.5% strength silver nitrate solution for 30 minutes.

4. Washing Step:

After removing the silvering solution, the gels are washed with Milli-Q water.

5. Development:

The washing solution is removed and the gels are developed with the developing solution for approx. 10-20 minutes, until the desired staining intensity is achieved. The developing solution consists of 1.4% sodium carbonate, 0.06% EDTA, 240 μl of 10% sodium thiosulfate solution and 800 μl of 37% strength formaldehyde solution, in each case per liter.

6. Stopping the Staining Process:

After development, the developing solution is removed and replaced with a stopping solution which is put from 1.5% EDTA solution or from a solution consisting of 5% Tris base and 2% acetic acid. The stopping step takes 5 minutes.

2. Staining Method According to Hochstrasser

The biomolecules to be stained are on polyacrylamide gels and are agitated on a horizontal shaker during the entire staining process. The solutions may be changed between the individual staining steps, for example, by removing with suction the no longer needed solutions and adding fresh solutions or by transferring the gels to new staining dishes.
1. Fixer 1 (40% ethanol+10% acetic acid)
   Duration of fixing process: 1h
2. Fixer 2 (5% ethanol+5% acetic acid)
   Duration of fixing process: 2 h or overnight
3. Washing step with Milli-Q water
   Duration: 5 minutes
4. Fixer 3 (74 g of sodium acetate trihydrate per liter+20 ml of 50% glutaraldehyde per liter)
   Duration of fixing process: 30 minutes
5. 3 washing steps with Milli-Q water
   Duration: 30 times 10 minutes
6. Incubation of the gels in naphthalene 2,6-disulfonic acid
   Duration: 30 minutes
7. Incubation of the gels in naphthalene 2,6-disulfonic acid
   Duration: 30 minutes
8. 4 washing steps with Milli-Q water
   Duration: 4 times 15 minutes
9. Silvering step
   After removing the washing solution (Milli-Q water), the gels are incubated in a silver diammine solution for 30 minutes.
   The silver diammine solution consists of 8 g of silver nitrate per liter, 13.3 ml of 25% ammonia and 4 ml of 5 M NaOH.
10. Four washing steps with Milli-Q water
   Duration: 4 times 4 minutes
11. Developing step
   The washing solution is removed and the gels are developed with the developing solution for 5-10 min.
   The developing solution consists of 100 mg of citric acid and 1 ml of 37% formaldehyde per liter.
12. Stopping step
   The developing solution is removed and replaced with a stopping solution.
   The stopping solution consists of 50 g of Tris base with 20 ml of acetic acid.
   Duration: 5-10 minutes
      3. Amersham Biosciences Kit

Using the Plus One Silver Stain Kit (protein), detection was carried out according to the manufacturer's protocol.

4. SYPRO Ruby

The SYPRO Ruby kit from Bio-Rad (# 170-3125) was used for fluorescent labeling and detection was carried out using an imaging system from Raytest (Fuji FLA 2000) according to the manufacturer's protocol.

5. Colloidal Coomassie

The biomolecules to be stained are in polyacrylamide gels and are agitated on a horizontal shaker during the entire staining process. The solutions may be changed between the individual staining steps, for example, by removing with suction the no longer needed solutions and adding fresh solutions or by transferring the gels to new staining dishes.

The colloidal Coomassie solution consists of: 2 g of Coomassie G250, dissolved in 1 l of Milli-Q water+55.5 ml of 95-97% sulfuric acid. The solution is stirred overnight and then filtered through a filter. Subsequently 220 ml of 10 M NaOH and 310 ml of 100% trichloroacetic acid are added.
1. Fixing and staining the gels with the colloidal
   Coomasssie solution
   Duration: overnight
2. washing step with Milli-Q water
   The staining solution is removed, followed by washing several times with Milli-Q water. The washing step is carried out until the gel background has been reduced to a minimum.

1. A method for detecting molecules, in particular peptides, proteins, carbohydrates, glycoproteins, proteoglycans and nucleic acids, by means of a metal compound in the presence of at least one at least bifunctional agent, said agent having at least one hydrophobic moiety and at least one reducing moiety.

2. The method as claimed in claim 1, wherein the bifunctional agent is a molecule of the general formula X—R.

3. The method as claimed in claim 1, wherein X is the reducing moiety.

4. The method as claimed in claim 2, wherein X is a linear or homo- or heterocyclic hydrocarbon.

5. The method as claimed in claim 2, wherein X preferably comprises at least one hydroxyl group, at least one sulfhydryl group, at least one carbonyl group, at least one thiosulfate group or at least one unsaturated carbon-carbon bond.

6. The method as claimed in claim 2, wherein X is a molecule having antioxidative properties, for example a vitamin, preferably from the group consisting of vitamin A, vitamin C or vitamin E, in particular ascorbic acid.

7. The method as claimed in claim 2, wherein R is the hydrophobic moiety.

8. The method as claimed in claim 2, wherein R is a saturated or at least monounsaturated hydrocarbon, preferably an acyloxy, amyl or alkyl radical.

9. The method as claimed in claim 2, wherein R is the acyloxy radical of the general formula —O—CO—CₙH₍₂ₙ₊₁₎, where n=8-21, preferably n=11-17, in particular n=15.

10. The method as claimed in claim 1, wherein the bifunctional agent is ascorbyl palmitate (=palmitoyl ascorbic acid), ascorbyl stearate (=stearoyl ascorbic acid), ascorbyl myristate (myristoyl ascorbic acid) or ascorbyl laurate (lauroyl ascorbic acid).

11. The method as claimed in claim 1, wherein the bifunctional agent is present at a final concentration of from 10⁻⁵ to 1% (w/v), preferably from 10⁻⁴ to 0.1% (w/v), in particular 5×10⁻⁴ to 5×10⁻³ (w/v) and preferably 10⁻³% (w/v), during detection.

12. The method as claimed in claim 1, wherein the metal compound is a silver compound, preferably silver nitrate.

13. The method as claimed in claim 1, wherein the nucleic acids are DNA or RNA.

14. The method as claimed in claim 1, wherein the molecules are applied onto or into a support for detection.

15. The method as claimed in claim 14, wherein the support is a gel, in particular a polyacrylamide or agarose gel, a membrane, in particular a PVDF or nitrocellulose membrane, or a microarray support, in particular a biochip.

16. The method as claimed in claim 14, wherein detection of the molecules, in particular those present on or in the support, comprises at least the following steps: fixing step, at least one washing step, metal compound step, developing step or stopping step.

17. The method as claimed in claim 16, wherein the bifunctional agent is used in the fixing step and, in particular, is present in a fixing solution.

18. The method as claimed in claim 14, wherein the bifunctional agent is used in an at least partially alcoholic solution, preferably as a component of the fixing solution, said alcohol preferably being ethanol.

19. The method as claimed in claim 14, wherein a complexing agent, preferably EDTA or EGTA, is used in the developing step and, in particular, is present in a developing solution.

20. The method as claimed in claim 19, wherein the developing solution comprises a reducing agent, preferably from the group of aldehydes, in particular formaldehyde, sodium carbonate, the complexing agent and/or sodium thiosulfate.

21. The method as claimed in claim 1, wherein the detected molecules are characterized, in particular studied mass-spectrometrically, after detection.

22. A kit for detecting molecules, comprising an at least bifunctional agent, said agent having at least one hydrophobic moiety and at least one reducing moiety, preferably in a fixing solution.

23. (canceled)

24. (canceled)
